(19) United States
(12) Patent Application Publication
(10) Pub. No.: US 2022/0057383 A1
(43) Pub. Date: Feb. 24, 2022

(54) USE OF BIOMARKERS TO EVALUATE THE EFFICACY OF A COMPOSITION IN REDUCING THE EFFECTS OF CANCER THERAPEUTICS ON SKIN

(71) Applicant: Johnson & Johnson Consumer Inc., Skillman, NJ (US)

(72) Inventors: Thomas Ondet, Val de Reuil (FR); Marius Monshouwer, Beerse (BE); Georgios N. Stamatas, Val de Reuil (FR); Pierre-Francois Roux, Val de Reuil (FR)

(21) Appl. No.: 17/084,106

(22) Filed: Oct. 29, 2020

Related U.S. Application Data
(60) Provisional application No. 62/950,624, filed on Dec. 19, 2019.

(51) Int. Cl. G01N 33/50

(52) U.S. Cl. CPC G01N 33/5014; G01N 33/5082

(57) ABSTRACT

A method to evaluate the potential of cancer therapeutics to produce skin related side effects is disclosed. The method, which involves use of biological markers, can also be used to evaluate the efficacy of a composition in reducing the effects of cancer therapeutics on skin.

This application claims priority of the benefit of the filing of U.S. Provisional Application Ser. No. 62/950,624, filed Dec. 19, 2019, the contents of which are hereby incorporated by reference herein in their entirety.

### FIELD OF THE INVENTION

The invention relates to methods to evaluate the potential of cancer therapeutics to produce skin related side effects. The present invention also relates to methods for evaluating the efficacy of a composition in reducing the effects of cancer therapeutics on skin.

### BACKGROUND OF THE INVENTION

Afatinib, a second generation EGFRi designed to overcome resistance following therapy with first generation drugs, was developed to irreversibly inhibit both the EGFR and the HER2 signaling pathways (Li et al. 2008). See structure below.

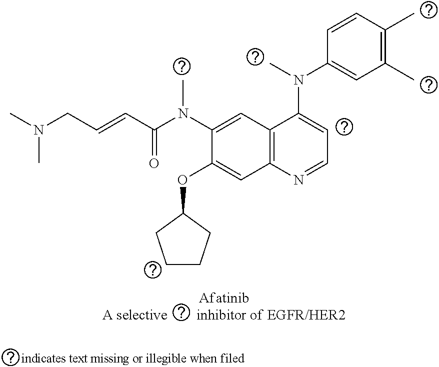

Even though the mechanism of drug-induced EGFR inhibition is largely understood, the mechanism resulting in the emergence of CADR remains unclear.

A candidate pathway for the survival of differentiating keratinocytes is the signaling pathway that combines Phosphoinositide 3-kinases (PI3K), a family of intracellular signal transducers with lipid kinase activity (Whitman et al. 1988), and the downstream serine-threonine kinase Akt effectors. The P13K/AKT complex elicits various cell responses involving cell growth, proliferation, differentiation and cell survival by controlling the anti-apoptotic mechanism (Calautti et al. 2005; Vivanco and Sawyers 2002). Downstream regulation of the PI3K/AKT pathway is mediated by several proteins, including the tumor suppressor Phosphatase and Tensin homolog (PTEN) (Ali et al. 2018) and caspase3 (Jänicke et al. 1998) that drive keratinocyte differentiation and eventual cornification rather than driving their apoptosis (Lippens et al. 2005). Other proteins, including the cyclin-dependent kinase (CDK) inhibitor p21Cip1/WAF1, retard cell growth and promote differentiation (Missero et al. 1996).

Vitamin D3 (VD3) or cholecalciferol is a factor that is often overlooked despite its critical role in epidermal development. VD3 is a secosteroid hormone synthesized in keratinocytes through UVB-induced photolysis via synthesis of 7-dehydrocholesterol, which results in the formation of its precursor (pre-VD3). The latter is then enzymatically hydroxylated to calcidiol by cytochrome P450 (CYP) and more specifically CYP2R1 and CYP27A1. Of note, pre-VD3 poorly regulates CYP2R1, contrary to CYP27A1 which is highly activated by pre-VD3 (Cheng et al. 2004; Mehlig et al. 2015). VD3 has a recognized impact on muscular, skeletal and immune physiology and is capable of promoting apoptosis and epithelial differentiation (Shaurova et al. 2020). CYP27A1 is also involved in the degradation of cholesterol in bile acids through both classic and acidic pathways (Norlin et al. 2003).

The development of polarized immune responses (involving the Th1/Th2 balance) is orchestrated by the activity of CD4+ Th cell subpopulations and their cytokine products. Th2 related cytokines, including interleukins (IL) 4, IL5, and IL13, lead to IgE production via an eosinophilic response by counteracting the Th1 mediated microbial action (Howell et al. 2008). Allergic inflammation in the skin results in Atopic Dermatitis (AD), a chronic disease characterized by intense pruritis, dryness and erythema in localized lesions (Bieber 2008). Th2 cytokine expression contributes to decreased expression of S100 proteins, which are involved in the regulation of proliferation, differentiation, apoptosis, Ca2+ homeostasis, energy metabolism, inflammation and migration/invasion, through interactions with a variety of target proteins including enzymes, cytoskeletal subunits, receptors, transcription factors and nucleic acids (Howell et al. 2008).

We previously reported that EGFRi promote keratinocyte differentiation and reduce proliferation (Joly-Tonetti et al. 2020). However, the molecular mechanisms associated with the CADR including skin rashes or more severe reactions, such as the Hand-and-Foot Syndrome or the Steven Johnson Syndrome (SJS) remain unclear. Afatinib, a second EGFRi generation drug, impairs the skin barrier structure by decreasing the epidermal thickness, reducing keratinocyte proliferation and increasing the expression of differentiation markers such as involucrin, filaggrin and desmoglein-1 (Joly-Tonetti et al. 2020).

### SUMMARY OF THE INVENTION

Phospho-proteomic and transcriptomic assays were conducted on Reconstructed Human Epidermis (RHE) tissues exposed to a therapeutically relevant concentration of afatinib to uncover the molecular signatures associated with CADRs.

Following drug exposure, activation of the PI3K/AKT pathway associated with an increased expression of gene families involved in keratinocyte differentiation, senescence, oxidative stress and alterations in the epidermal immune-related markers was observed.

The results show that afatinib may interfere with Vitamin D3 (VD3) metabolism, acting via CYP27A1 and CYP24A1 to regulate calcium concentration through the PI3K/AKT pathway. Consequently, basal layer keratinocytes switch from a pro-proliferating to a pro-differentiative program, characterized by upregulation of biomarkers associated with increased keratinization, cornification, Th2 response and decreased innate immunity. Such effects may increase the skin susceptibility to cutaneous penetration of irritants and pathogens.

Taken together these findings demonstrate a molecular mechanism of EGFRi-induced CADRs. These pathways provide new biomarkers to evaluate the efficacy of a composition in reducing the adverse reactions of therapies, including cancer therapy.

### DESCRIPTION OF THE FIGURES

FIGS. 1A-1D show functional perturbation in RHE keratinocytes following exposure to afatinib compared to control.

A generalized linear model accounting for the interaction between time and treatment was considered (Afatinib at 100 nM versus control).

FIG. 1A is a volcano plot depicting the results of the protein differential analysis performed on the phosphorylation array. Using a cut off of 1.2-fold change, 62 proteins with a change in their phosphorylation status were identified including 44 proteins with a significant increase (green) and 18 proteins with a significant decrease (red) of their phosphorylation status. In a time dependent manner, after 20 min of afatinib exposure the phosphorylation status of 6 proteins was increased and that of 3 proteins was decreased. At 24 h, an increased phosphorylation status was observed for 18 proteins and a decreased phosphorylation status for 23 proteins. At 72 h, the protein phosphorylation status was decreased for 6 proteins and increased for 6 others.

FIG. 1B is a volcano plot depicting the results of the differential analysis performed on gene expression (cut off: 50%). At 6 h, the expression of 170 genes was decreased and the expression of 201 genes increased. At 24 h, 335 genes were down- and 281 genes up-regulated. At 72 h, gene expression was increased for 2888 genes and decreased for 1728 genes.

FIG. 1C shows set Enrichment Analysis identified significant enrichment of the PI3K-AKT pathway at 24 h of afatinib exposure. Proteins with increased of their phosphorylation status are shown in green and proteins with decreased phosphorylation status are shown in red.

FIG. 1D shows the pathway associated to cellular senescence, involving irreversible growth arrest accompanied by phenotypic modification, showed a significant enrichment based on the change of protein phosphorylation status. The senescence pathway is downstream of the PI3K/AKT pathway.

FIGS. 2A-2D show that clustering reveals functional impact of afatinib on keratinocytes differentiation, oxidative stress and innate immune response.

FIG. 2A is a heatmap and FIG. 2B are boxplots depicting the results of k-means clustering based on the phosphorylation status during the time-course. Data in the heatmap is expressed as row Z-score. In boxplots, the center lines depict the median, the lower and upper edges of the boxes correspond to the first and third quartiles. The upper whisker extends from the edge of the box to the largest value up to 1.5× the interquartile range (IQR) from the edge, while the lower whisker extends from the box edge to the smallest value at 1.5× the IQR of the edge

FIG. 2C is a heatmap and FIG. 2D are boxplots depicting the results of k-means clustering based on the gene expression during the time-course. Genes were divided in 6 clusters to classify the change in genes expression considering treatment vs. control. Data in the heatmap is expressed as row Z-score. In boxplots, the center lines depict the median, the lower and upper edges of the boxes correspond to the first and third quartiles. The upper whisker extends from the edge of the box to the largest value up to 1.5× the interquartile range (IQR) from the edge, while the lower whisker extends from the box edge to the smallest value at 1.5× the IQR of the edge

FIGS. 3A and 3B show an over-representation analysis based on KEGG confirms functional impact of afatinib on keratinocytes differentiation, oxidative stress and innate immune response.

FIG. 3A and FIG. 3B show functional over-representation maps depicting KEGG gene sets associated to each phosphorylation (see FIG. 3A) and transcriptomic (see FIG. 3B) clusters. Dots are color-coded according to the FDR corrected p-value based on the hypergeometric distribution. Size is proportional to the percentage of genes in the gene set belonging to the cluster

FIG. 4 shows the EGFR signaling pathway and its dependencies. Afatinib inhibits EGFR but activates the PI3K/Akt pathway.

EGFR pathway activation triggers 3 different pathways: PI3K/Akt, RAS/RAF/ERK and STAT. Upon Afatinib treatment on RHE keratinocytes, the PI3K/AKT pathway is alternatively increased. Consequently, genes downstream of the PI3K/AKT pathway, involved in proliferation, survival and senescence were impacted resulting in skin barrier function impairment.

FIGS. 5A-5D show Protein Array quality check before and after normalization and filtering. FIG. 5A is a Box plot depicting the distribution of fluorescence signal before and after quantile normalization for the phospho-antibody microarray. FIG. 5B shows unsupervised bi clustering for the phosphorylation protein array before and after quantile normalization (distance: Pearson's correlation, aggregation criterion=Ward's D2). FIGS. 5C and 5D show Biplots for Principal Component Analyses, before normalization and after normalization and de-batching for the transcriptome assay. The quantile normalization followed by the de-batching step allow to capture time along PC1 and treatment along.

Supplementary Table S1: Up/down regulated phosphorylated protein. See attached Excel file.

Supplementary Table S2: Up/down regulated gene list. See attached Excel file.

### DETAILED DESCRIPTION OF THE INVENTION

### The Examples Show that Protein Phosphorylation Status and Gene Expression in RHE Keratinocytes Dynamically Change Following Exposure to EGFRi

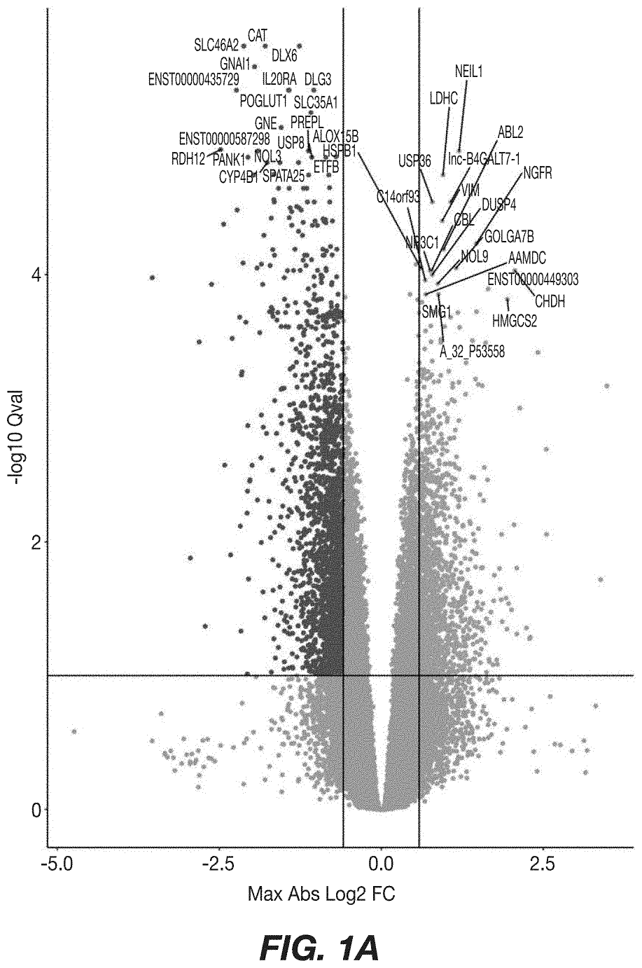

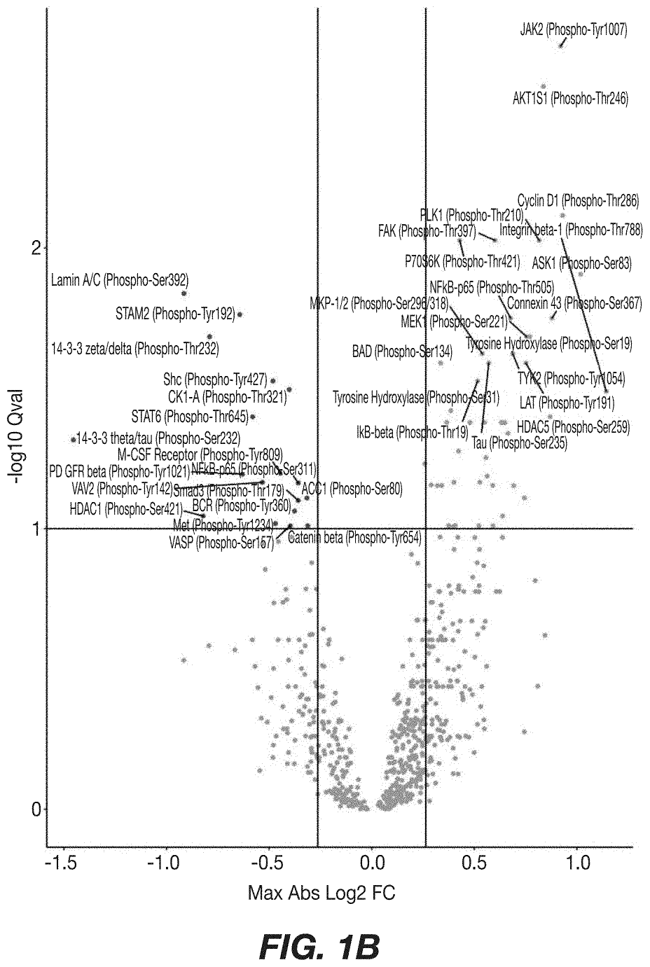

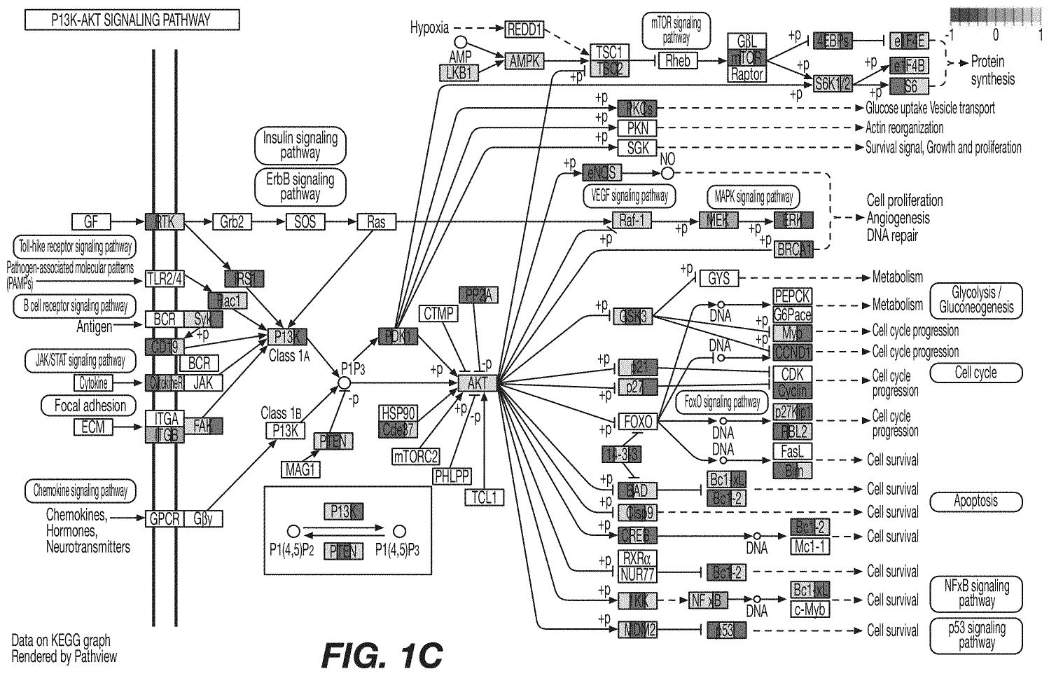

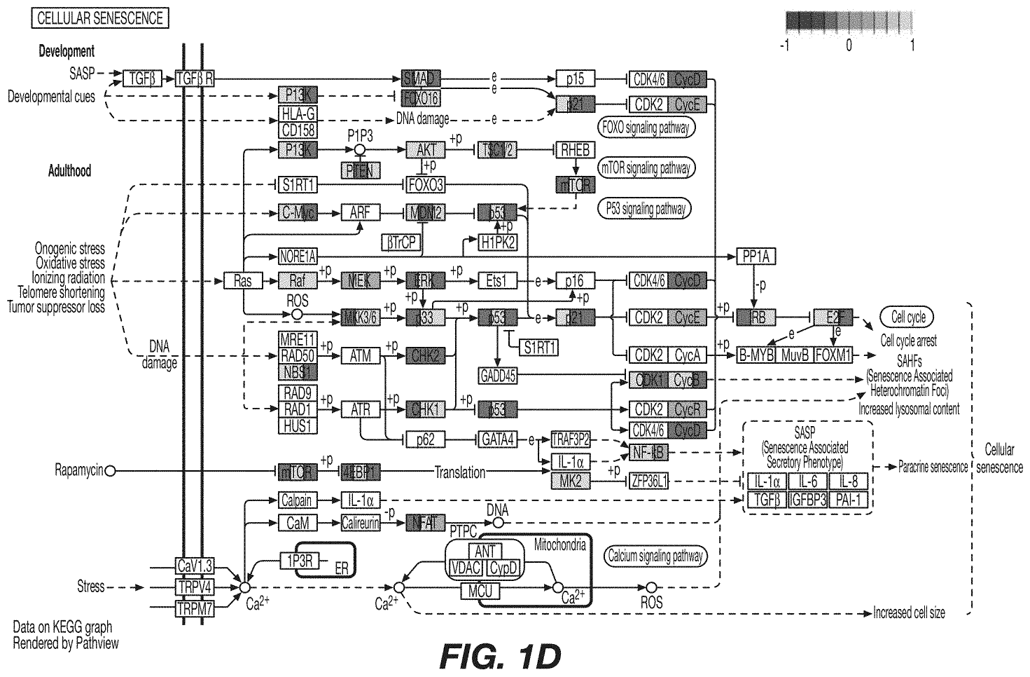

A phospho-antibody microarray was used to identify differences in the phosphorylation status of proteins from the RHE tissues following exposure to 100 nM afatinib for 20 min, 24 h and 72 h. In this assay, 1318 proteins including 615 phosphoproteins were screened and changes in their phosphorylation status were recorded at each time point (Supplementary Table 51). A total of 62 phosphoproteins changed their phosphorylation status by more than 20% (FIG. 1A). Following exposure to afatinib for 20 min, 24 h and 72 h, the number of proteins showing increased phosphorylation compared to control was respectively 6, 18 and 6 and the number of proteins showing decreased phosphorylation compared to control was respectively of 3, 23 and 6.

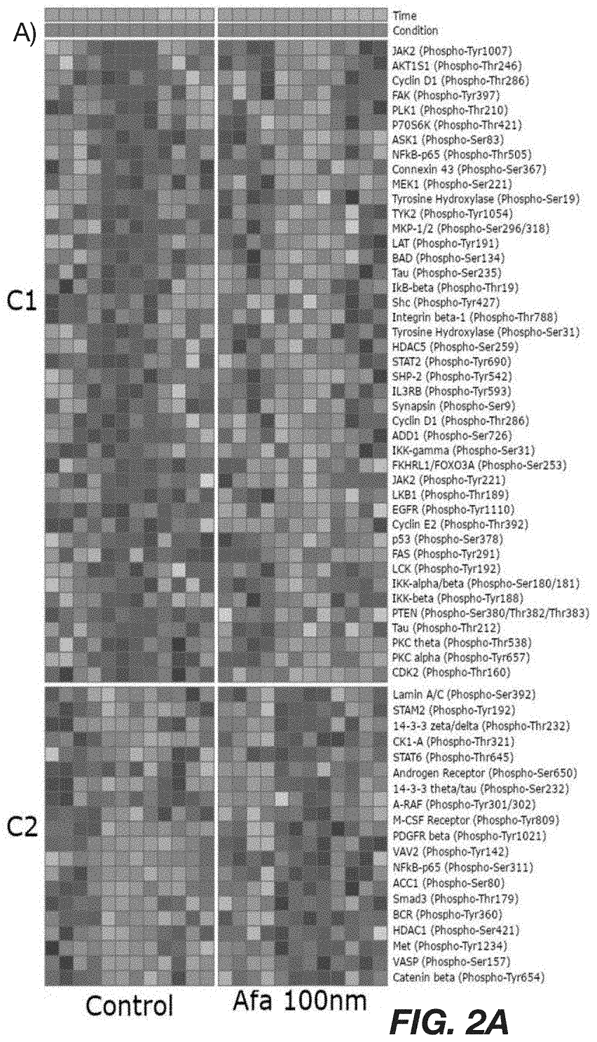

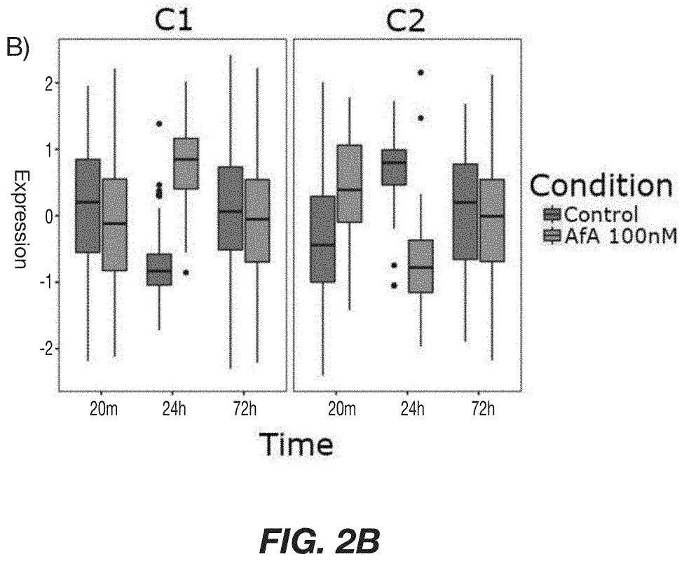

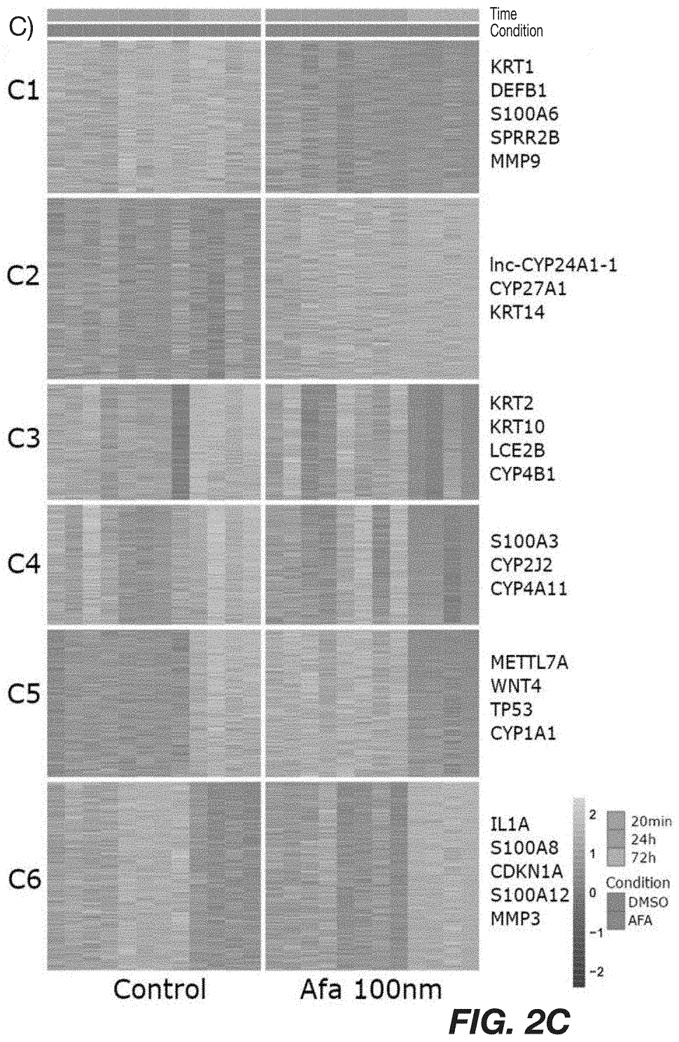

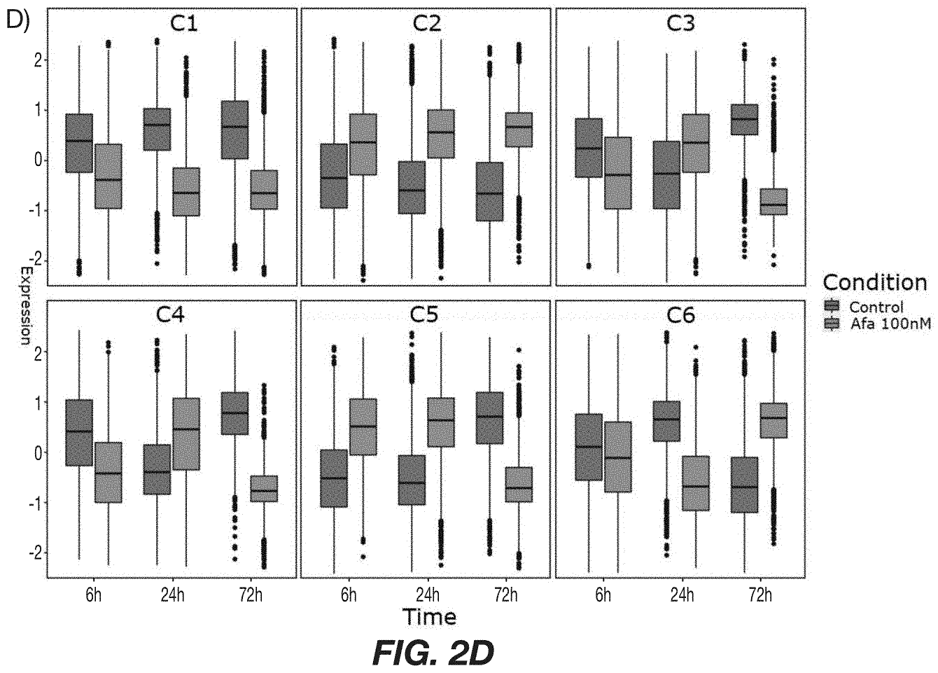

Unsupervised clustering was performed to categorize the proteins into clusters (C) depending on the level of alteration of their phosphorylation status and the timepoint when the event was detected. Such clustering allows for a more precise determination of the biological functions, interactions or pathways that are enriched. After defining the optimal number of clusters, the proteins were grouped into two clusters, the first one (C1) representing proteins with decreased levels of phosphorylation at 24 h, and the second (C2) representing those with increased levels of phosphorylation at 24 h (FIGS. 2A and 2B).

Transcriptomic analysis of RHE keratinocytes treated with afatinib was assessed by screening for the expression of 36,000 genes and long non-coding RNA (IncRNA) (Supplementary Table S2). A cut-off at 50% in the fold-change expression revealed the dysregulation of 2,182 genes at different time points. After 6 h exposure to 100 nM afatinib, 170 genes were down-regulated compared to control and the 201 genes were up-regulated. At 24 h exposure, the expression of 335 genes was decreased and the expression of 281 genes was increased. At 72 h exposure, the expression of 2,888 genes was decreased and the expression of 1,728 genes was increased (FIG. 1B).

Unsupervised clustering was performed for the transcriptomic data to categorize the genes into clusters depending on changes in their expression levels following exposure to afatinib and the timepoint when the event was detected. After defining the optimal number of clusters, the data were grouped as follows (FIGS. 2C and 2D):

C1, representing early (20 min) downregulated genes that maintain their status at all three time points tested; C2, representing the early (20 min) upregulated genes that maintain their status at all three time points; C3, representing transiently upregulated genes at 24 h; C4, representing transiently upregulated genes at 24 h with higher amplitude compared to C3; C5, representing early upregulated genes followed by downregulation at 72 h and C6, representing downregulated genes at 24 h followed by upregulation at 72 h.

### Protein Phosphorylation Array Analysis Demonstrates Activation of the PI3K/AKT Pathway with Implications on Cellular Senescence

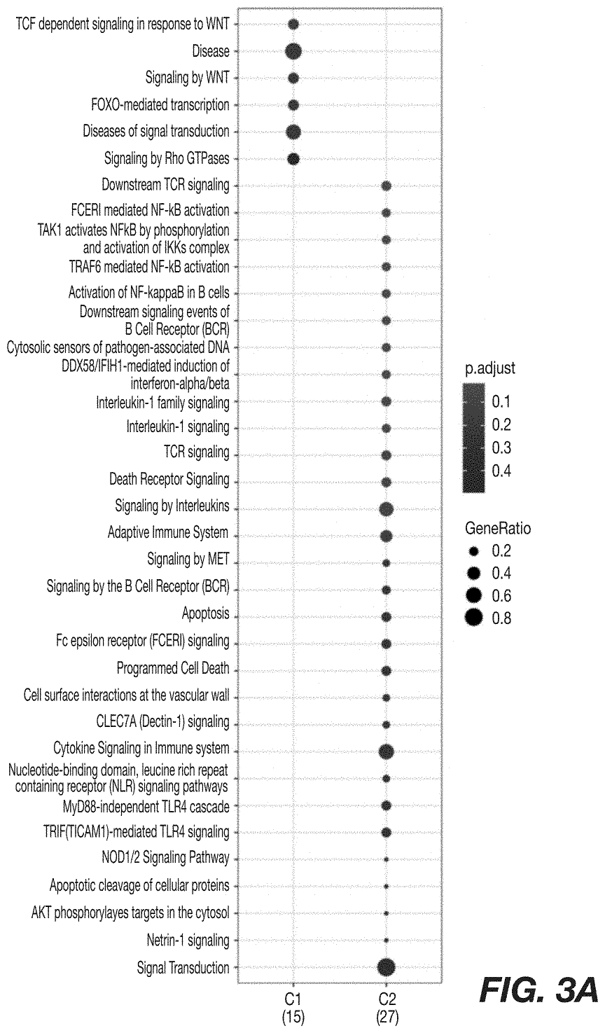

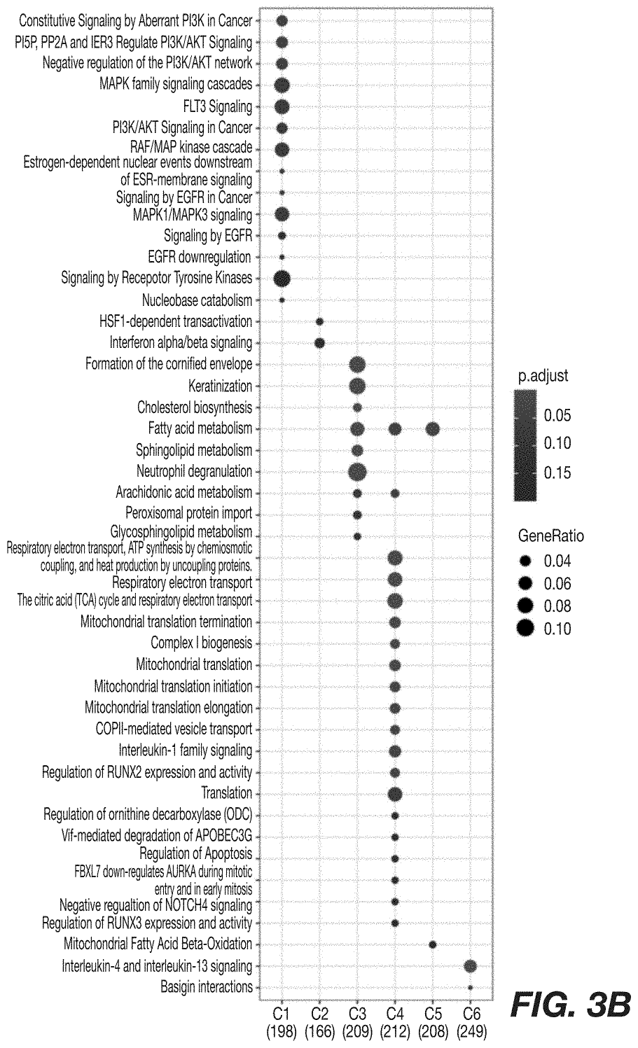

Based on the alterations of the phosphorylation status, the Set Enrichment Analysis (SEA) identifies the PI3K/AKT pathway as the main enriched pathway (FIG. 1C) and more specifically the senescence pathway (FIG. 1D). Upstream of PI3K, proteins with increased phosphorylation included RAC1 and FAK. Downstream of PI3K, p21 (CDKN1) is involved in cell cycle progression, BAD in apoptosis and p53 in cell survival. Proteins with decreased phosphorylation are mostly inhibitors, such as PTEN, GSK3 and BCL2, and consequently promote activation of this pathway. Interestingly, an important number of regulated proteins downstream of the PI3K/AKT pathway are involved in cellular senescence by activating FOXO3, the cyclin gene family (CYCB, CYCD, CYCE) but also the mTOR pathway and dephosphorylation of 4EBP1. The functional over-representation test showed an increased activity of proteins related to AKT phosphorylation (FIG. 3A).

The transcriptomic assay results confirm activation of the PI3K/AKT pathway, as indicated by the upregulation of genes related to this pathway (C6, FIG. 3B).

### Gene Expression Array Analysis Highlights CYP as Potential Oxidative Stress Inducer for Keratinocyte Differentiation

Both protein phosphorylation and gene expression arrays demonstrated that gene families involved in cellular stress, oxidative stress and senescence were impacted following exposure to afatinib (FIG. 3). For example, FOXO3 phosphorylation was decreased as a consequence of the PI3K/AKT pathway activation. The FOXO3 transcription factor affects the cell capacity to regulate apoptosis and programmed cell death.

Afatinib dynamically affected the expression of genes related to oxidative stress. A large panel of metallothionein (MT1E, MT1L, MT1H L1, MT1X, MT1B, MT1A) was decreased at 24 h and increased at 72 h (FIG. 3B). Gene families involved in the respiratory electron transport and mitochondrial translation termination were transiently upregulated at 24 h (FIG. 3B). More specifically, the expression of the antioxidant enzymes PRDX2 and PRDX3 was decreased and the expression of SIRT4 was increased at 72 h (Supplementary Table S2). Of note, genes related to the metabolism of fatty acids associated with oxidative stress were firstly upregulated at 24 h and then downregulated at 72 h (FIG. 3B).

Interestingly, changes in CYP expression at 24 h and 72 h were also identified. The expression of CYP was increased (Inc-CYP24A1-1, CYP27A1, CYP4F12) and the expression of others CYP was decreased (CYP4B1, CYP3A7, CYP3A5, CYP2J2, CYP4A11, CYP1A1, CYP4F22) (Supplementary Table S2). CYP27A1 expression could be associated with transcriptomic data involving cholesterol biosynthesis and fatty acid metabolism in cluster C1.

Besides oxidative stress-related genes, the functional overrepresentation test (FIG. 3) showed enrichment in keratinization and cornification pathways, associated to the increase at 24 h in the expression of KRT2, KRT9, KRT13, KRT15 and Late Cornified Envelop (LCE) genes (LCE2A, LCE1B, LCE2B, LCE1C, LCE2D).

### Exposure to Afatinib Perturbed the Expression of Innate Immunity Markers and Enhanced the Production of Cytokines Related to the Th2 Response

The PI3K/AKT pathway directly impacts both innate and adaptive epidermal immunity. Analysis of the transcriptomic data showed that genes involved in the innate immunity were negatively regulated (Supplementary Table S2) including genes related to the S100 family (S100A2, S100A6, S100A7, S100A8, S100A9, S100A10, S100A12), Small Proline Rich Protein 2B (SPRR2B) and the P-defensin B1 (DEFB1) (FIG. 3A).

The gene family associated to the Th2 response was affected as demonstrated by the over representation of genes related to the IL4 and IL13 signaling (C2). Interestingly, these results show the kinetics of the consequences following PI3K activation, which involve first activation of TCR signaling at 24 h and later the Th2 polarization at 72 h.

On the other hand, inflammation pathways associated to the Th1 response were barely activated. The transcriptomic data did not show any increase in expression of genes related to inflammation. The protein phosphorylation assay and the gene expression array showed a decreased in inflammasome response via TLR, NLR and IL1 families.

### Discussion

The majority of the clinically reported skin symptoms of patients treated with small molecule EGFRi include rashes (63% of patients), xerosis (30%) and granulomas (30%). No relationship has been reported between CADR symptoms and specific EGFRi apart from a more frequent appearance of granulomas in afatinib-treated patients (Annunziata et al. 2019). These data reinforce the pertinence of afatinib as a comprehensive model drug in the study of CADRs.

Besides small molecule treatments, immunotherapies exist that target the extracellular part of the EGFR. Although monoclonal antibodies (mAB) have shown less off-target effects, they can still induce severe skin rashes (Tischer et al. 2017) that are typically associated with activation of immune system components. By focusing on the direct effects of oncology therapy on epidermal physiology and its consequences on skin barrier function, in our model we intentionally removed effects related to an over activated immune system.

Moreover, to better focus on the effects of afatinib besides its known action as EGFRi, exogenous EGF was not included in the media. The presence of EGF associated with afatinib would have highlighted the drug's consequences downstream of the EGFR.

Maintaining keratinocytes in a proliferative phenotype requires specific cell culture conditions. Any modification to the cellular metabolism rapidly induces keratinocytes to differentiate (Bakondi et al. 2003; Tsuchisaka et al. 2014; Vessey et al. 1995; Zhang et al. 2002). Exposure to afatinib may impact epidermal formation through a single or a combination of alterations in keratinocyte metabolism.

### The PI3K-AKT Pathway Promotes Keratinocyte Senescence and Suppresses Keratinocyte Proliferation

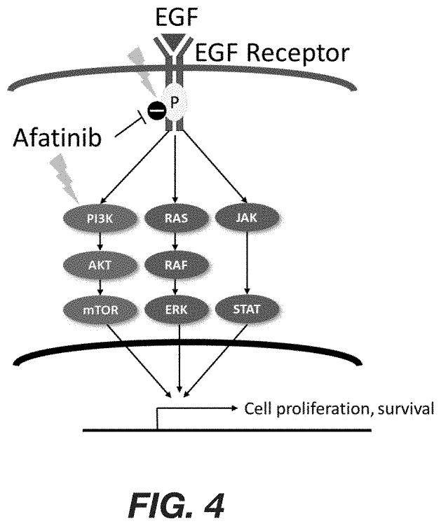

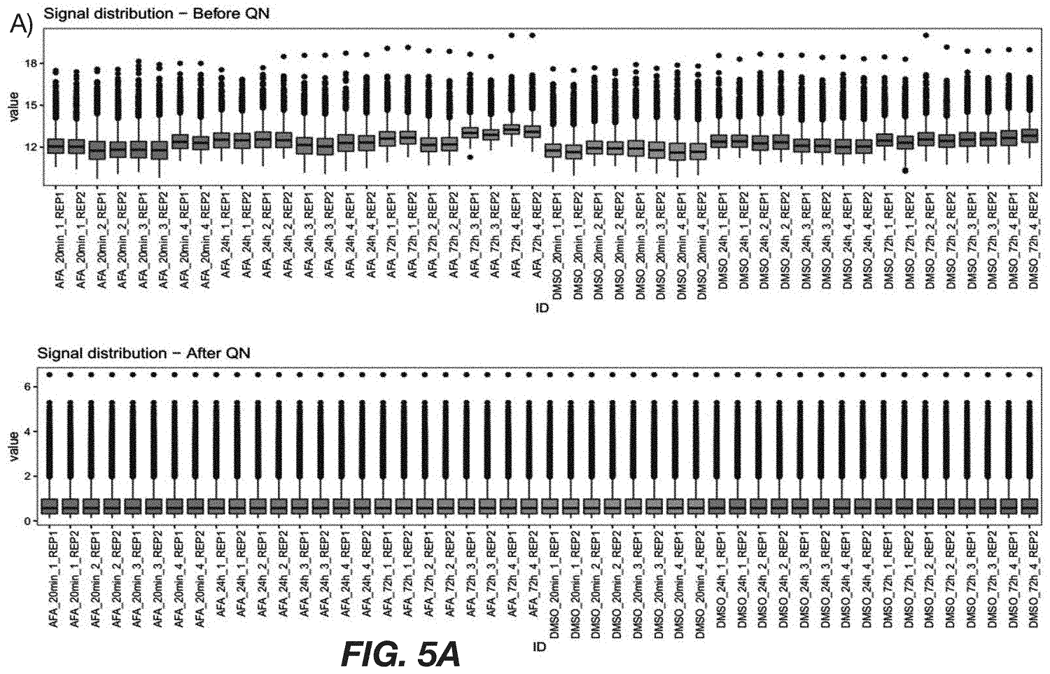

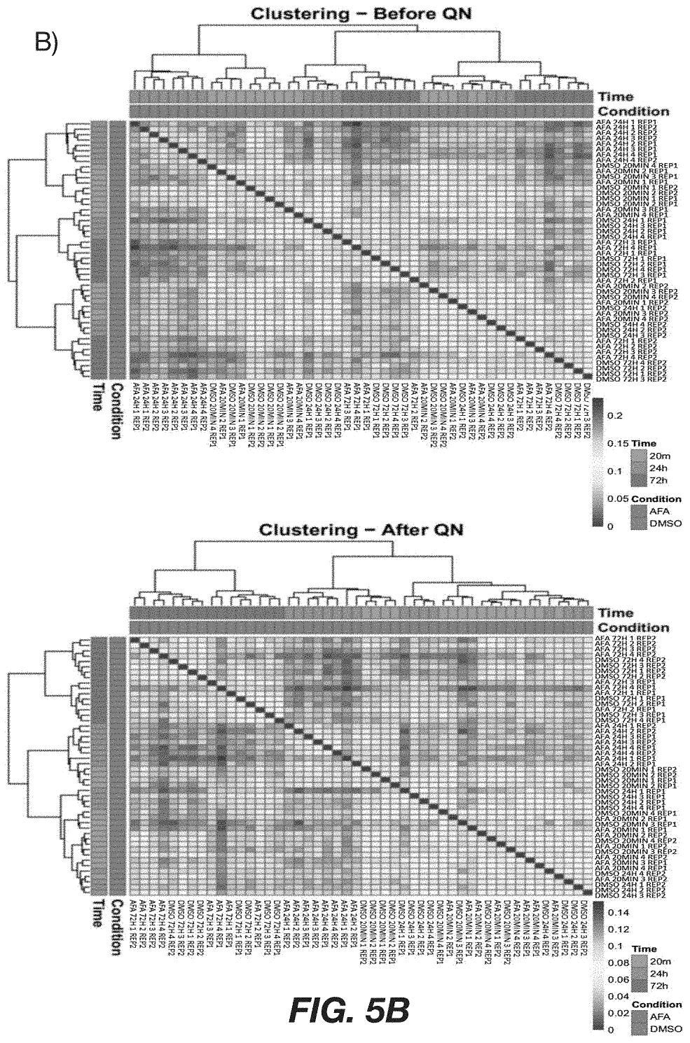

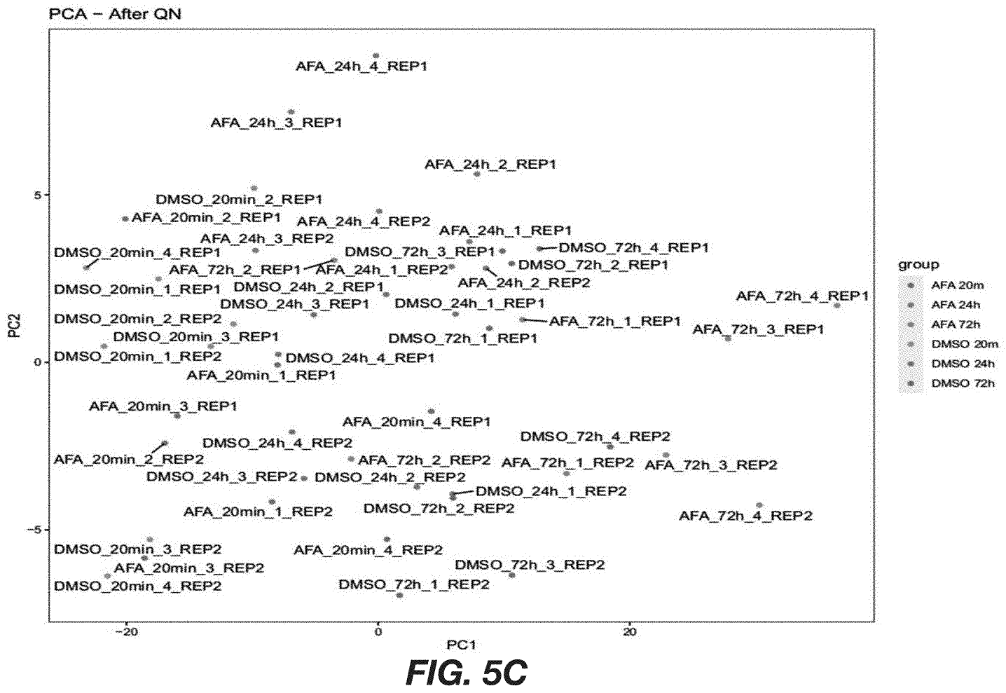

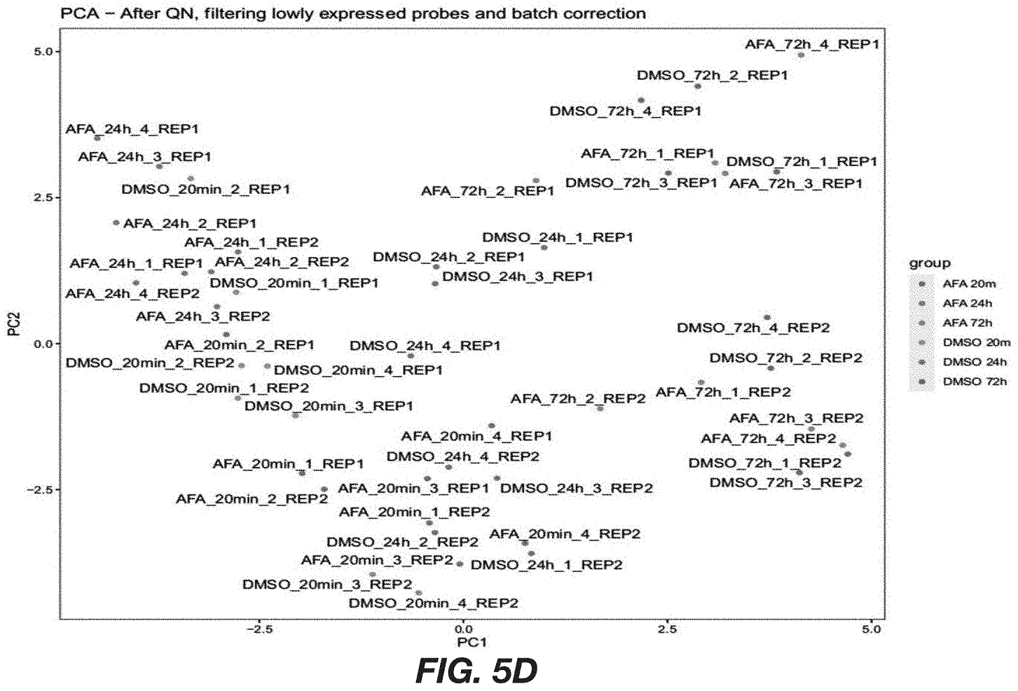

Following keratinocyte exposure to afatinib there was a significant increase of activity related to the PI3K/AKT pathway. This was surprising, since this pathway is downstream of the EGFR (FIG. 4) and is expected to be negatively regulated by an EGFRi.

Although the PI3K/AKT pathway was upregulated, the RAF/RAS/ERK1/2 pathway, which is another independent pathway downstream of EGFR activation, was not positively activated following treatment with afatinib at 24 h (FIG. 4) (Wee and Wang 2017). The phosphorylation of Raf1 (Phospho-Ser338), eIF4E (Phospho-Ser209) were decreased and the phosphorylation of Raf1 (Phospho-Tyr341), MEK1 (Phospho-Ser221), MEK1 (Phospho-Ser217) were not impacted (Supplementary Table S1). These results showed the expected effect of afatinib downstream of EGFR. We hypothesize that the PI3K/AKT activation was the consequence of an alternative mechanism.

The PI3K/AKT pathway impacts keratinocyte gene expression related to cell growth, cell differentiation, senescence and apoptosis, and increased oxidative stress leading to differentiation. Afatinib impacts skin barrier formation by inducing inflammation and decreasing the innate immune response (Calautti et al. 2005; Janes et al. 2009). Our results showed that the senescence pathway (FIG. 1D) was specifically activated through the involvement of PUK, C-Myc, p53 and mTOR proteins. Increased activity in the regulation of these proteins is known to directly induce cell cycle arrest and promote senescence (Demidenko et al. 2010; Iglesias-Bartolome et al. 2012).

We previously reported that the expression of epidermal differentiation markers was increased following afatinib exposure (Joly-Tonetti et al. 2020). Interestingly, the PI3K/AKT pathway regulates the increase of filaggrin, loricrin and cadherin-associated catenin (Calautti et al. 2005) which confirms our previous observations.

Moreover, activation of the PI3K/AKT pathway favors a decrease of cell attachment leading to apoptosis (Janes et al. 2009) and results in an increase of differentiation marker expression and consequently induce keratinocytes differentiation. Genes associated with terminal differentiation were transiently increased at 24 h and decreased at 72 h of exposure to afatinib. These results indicate that afatinib induces a rapid induction of keratinocyte differentiation and could be the consequence of the increased activation of PI3K.

In summary, activation of the PI3K/AKT pathway promotes keratinocytes differentiation and suppresses keratinocytes proliferation. In certain cases, induction of apoptosis could be involved in more severe conditions such as in the Hand-Foot Syndrome.

### Afatinib Induces Cellular Oxidative Stress which Triggers Keratinocyte Differentiation

Upon exposure to afatinib the expression pattern of several proteins downstream of AKT was altered, including decreased FOXO3A expression, a protein known to be directly downregulated by AKT (Brunet et al. 1999) and consequently increased expression of Cyclin D1 (CDKN1), which is linked to oxidative stress (Marinkovic et al. 2007).

The p53 protein plays a central role in regulating the keratinocyte life cycle: it controls the rate of cell proliferation and favors differentiation over apoptosis. Active p53 triggers expression of well-established pro-senescence targets. The p21 protein controls the pathways linked to cellular aging and senescence (Herbig et al. 2008). In this regard, increased p53 levels are able to limit the oxidative damage and participate in proapoptotic and pro-senescent activities (Rufini et al. 2013). Nevertheless, our phospho-antibody microarray results do not show an increase in Caspase 3 activity (Supplementary Table S1) at any time point which also confirms our previous results (Joly-Tonetti et al. 2020) that mild to moderate CADRs are not associated to keratinocyte apoptosis. However, in more severe conditions such as TEN, p53 activation could be the cause of more serious symptoms (Ido Y. 2015).

### EGFRi Impact Vitamin D3 Metabolism in Keratinocyte

In the presence of EGF, afatinib irreversibly inhibits EGFR intracellular signaling by covalently binding to Cys797 of the EGFR, Cys805 of HER2 and Cys803 of ErbB-4 (Solca et al. 2012). An off-target activity of the drug has already been demonstrated through nonspecific covalent binding (such as to CDK complexes) leading to disturbance of cellular processes (Klaeger et al. 2017). These results suggest that covalent kinase inhibitors have the potential to cross-react, either specifically or nonspecifically, with proteins outside their kinome. Such activity complicates the assignment of biological functions to kinases in chemical biology experiments and could lead to unanticipated toxicities by triggering apoptosis and/or senescence (Lanning et al. 2014). Off-target binding of TKi has been shown to enhance calcium metabolism and contribute to keratinocyte differentiation (Kroschwald et al. 2018).

A parallel activation of vitamin D3 (VD3) metabolism may consequently interfere with extracellular calcium levels to induce keratinocytes differentiation (Teichert and Bikle 2011). A recent study (Shaurova et al. 2020) has shown a direct impact of EGFRi on 1,25-dihydroxyvitamin D3 (1,25(OH)2D3) (1,25(OH)2VD3), a VD3 metabolite involved in epithelial differentiation.

Our results show that following afatinib exposure, the expression of several CYPs was altered. These include CYP27A1 involved in VD3 metabolism and the degradation of cholesterol to bile acids in both the classic and acidic pathway. A previous study (Kroschwald et al. 2018) has shown that TKi directly modulate CYP24A1 activity. As a consequence, a change in calcium metabolism takes place that could impact keratinocyte differentiation (Elsholz et al. 2014).

Moreover, low levels of a key vitamin D catabolizing enzyme 24-hydroxylase has been correlated with the CYP24A1 gene expression (Shaurova et al. 2020). Interestingly, our results show an increase of Inc-CYP24A1 and consequently suggest a decreased in CYP24A1 expression. Both of these CYPs has been shown to induce calcium metabolism modification (Dinour et al. 2013). Furthermore, 1,25(OH)2D3 induced transcriptional responses including increased mRNA levels of CYP24A1, a well-characterized direct target of the Vitamin D receptor (VDR) (Shaurova et al. 2020). All together, these results showed enhanced expression of 1,25(OH)2D3 associated to an upregulation of CYP24A1, both of which promote epithelial differentiation.

1,25(OH)2D3 is known to induce transcription of multiple cell adhesion molecules (Palmer et al. 2001) and confirms our previous results showing that afatinib affects epidermal size and volume and increases the expression of differentiation markers (involucrin, desmoglein and filaggrin) (Joly-Tonetti et al. 2020). In relation to oxidative stress, VD3 is known to be a potent inducer of metallothionein (MT), able to capture harmful oxidants, such as the superoxide and hydroxyl radicals (Nzengue et al. 2008) (FIG. 3B). MT may act as a radical scavenger in oxygen mediated CYP activity.

Interestingly, studies have demonstrated that a deregulation of the PI3K/AKT pathway synergizes with the antiproliferative signaling of VD3 to induce cell senescence (Axanova et al. 2010; da Silva Teixeira et al. 2020) and confirmed both the involvement of VD3 and the senescence pathway as potential mechanisms of CADRs.

TKi in general, and specifically afatinib, are known to induce photosensitivity (Dai et al. 2017). It is very likely that exposure to solar UV radiation may interact with such therapies affecting the VD3 metabolism and relating to increased skin photosensitivity.

### Immune Alterations Following EGFRi Treatment Compromises the Skin Barrier Protection

The mechanisms through which the immune response is affected by TKi therapy are still poorly understood. In this work, protein and gene expression assays demonstrated the involvement of the Th2 response as a result of a combined increase of related IL-4 and IL-13 gene expression, as well as a decrease of both the Th1 response and the innate immune response via S100 proteins and the inflammasome (Howell et al. 2008). Interestingly, the same changes are observed in atopic dermatitis (Howell et al. 2008). Moreover, a dysregulated VD3 metabolism impacts negatively the immune response in keratinocytes (Kroschwald et al. 2018).

The impaired epidermal function associated with the impaired innate immune response increases the susceptibility of individuals to recurrent bacterial and viral skin infections, that are often clinically observed. Use of tetracycline to compensate for the impaired innate immune response following afatinib exposure has shown improvement of the CADR symptoms (Arrieta et al. 2015).

These new markers could be useful in designing better strategies for prevention of skin damage resulting from oncology therapies.

To conclude, this work demonstrates that the PI3K/AKT pathway plays a pivotal role in the pathogenesis of CADRs by quickly driving keratinocytes differentiation. This mechanism could explain the manifestation of CADR symptoms. These results also indicate that VD3 plays an important role in CADRs. TKi are known to impact VD3 metabolism. A deregulated VD3 pathway impacts keratinocytes differentiation through alterations in intracellular calcium activity, increased differentiation protein expression, decreased innate response and an overexpression of CYP21A1. Taken together, these results bring new molecular insights in the mechanisms of CADRs that can be useful in clinical diagnosis as well as assessment of the efficacy of a CADRs treatment protocol.

### Materials and Methods

### Drug Preparation

The afatinib concentration used in the experiments was selected at a sub-cytotoxic level of 100 nM, as previously reported (Joly-Tonetti et al. 2020). Afatinib was purchased from Caymanchem (Ann Arbor, Mich., USA) and was prepared from a 10 mM stock solution dissolved in DMSO (Sigma, St. Quentin Fallavier, France). Consequently, the final DMSO concentration was 0.001% for 100 nM. The control was composed of the same DMSO volume as the afatinib solution.

### Reconstructed Human Epidermis Model

Large 4 cm² RHEs were purchased from Episkin (Lyon, France) and were cultured for 24 h without EGF before exposure to afatinib. RHE treatment was performed in Epilife medium without EGF and without proteins (MEPI500CA, Thermo Fisher Scientific, Waltham, Mass., USA). A short, intermediate and long drug exposure times were selected of respectively 20 min, 24 h, 72 h for the protein phosphorylation assay and 6 h, 24 h and 72 h for the gene profiling assay. Quadruplicate repeats were performed for all timepoints and conditions.

### Antibody Microarrays for Protein Phosphorylation and Expression Profiling

The Phospho Explorer Antibody Array (Full Moon BioSystems, Inc., Sunnyvale, Calif., USA) was performed by TebuBio (Le Perray-en-Yvelines, France) using 1330 duplicate spots matching 1318 proteins (phosphorylated and unphosphorylated), house-keeping proteins (Beta actin, GAPDH), negative controls (n=4), empty spots (n=4) and positive markers (n=2) were analyzed. Briefly, RHEs were washed in cold PBS and directly stored at −80° C. The samples were treated following the manufacturer's protocol. Sample volumes of 60 μg were incubated with biotin for 2 h followed by 30 min incubation in the Stop reagent (Full Moon BioSystems, Sunnyvale, Calif., USA). Membranes containing printed antibodies were blocked for 40 min with the Blocking reagent under agitation, then they were washed and incubated in a coupling chamber for 2 h. After sample removing and three successive washes, the detection step was performed after addition of 30 μl of Cy3-Streptavidin (1 mg/ml) for 20 min incubation at room temperature in the dark. The slide was then washed, dried by centrifugation and scanned on a microarray scanner (Innopsys Innoscan 710).

### Gene Expression Profiling

Transcriptomic analysis was performed by Genex (Longjumeau, France). RHEs were removed from their supporting polycarbonate membrane and immediately transferred in RLT buffer containing 1% β-mercaptoethanol. Polycarbonate membranes were scraped to remove attached remained basal cells and transferred in the corresponding vials containing the RHEs. Samples were immediately stored at −80° C.

Total RNA including miRNA was extracted using AllPrep DNA/RNA/miRNA Universal kit from Qiagen (Hilden, Germany). RNA quality was assessed by Experion (Biorad, Marnes-la-Coquette, France). All RNA quality indicators were above 7. A quantity of 100 ng of total RNA was transcribed and stained with cyanine 3 (Cy3) using RNA Low Input Quick Amp Labeling Kit One Color (Agilent Technologies, Les Ulis, France) according to the manufacturer's instructions. All specific activity was above 6 pmol Cy3/μg cRNA and the yield above 1.65 μg. An equal amount (600 ng) of Cy3 labeled cRNA was fragmented and subsequently hybridized for 17 hrs at 65° C. onto human SurePrint 8x60K v3 microarray (Agilent Technologies). The microarrays were then washed and scanned according to the manufacturer's instructions by Scanner G2505C (Agilent Technologies). Gene expression data were further processed using Feature Extraction (version 10.7) software.

### Data Processing

For both protein phosphorylation and transcriptome array data, a background correction followed with a quantile normalization (QN) was performed using the RMA framework as implemented in the limma package. This procedure is aimed at normalizing the median expression between arrays (Supplementary FIGS. S1 and S2).

Quality-check was performed to properly evaluate the impact of the QN and filtering using a graphical data visualization before and after normalization, associated with Box plot, unsupervised clustering, Principal Component Analysis and Multidimensional Scaling (Supplementary FIGS. S1 and S2).

Volcano plots were determined based on a significant differential expression of −log10 of transformed q-values after applying a 10% false discovery rate followed by the Benjamini-Hochberg post analysis (Y axis). The X-axis indicates the difference between the two groups (Afatinib vs. control). Protein phosphorylation and gene expression values were log2 transformed to ensure normal distribution.

To identify the phosphorylation sites and genes impacted by afatinib at any time point, a differential analysis relying on the General Linear Model was performed, considering time and treatment interaction, using the limma package.

Gene and protein Set Enrichment Analysis (SEA) was then performed to identify classes of genes or proteins that are over-represented in genes sets that are up- or down-regulated and may have an association with disease phenotypes. The Kyoto Encyclopedia of Genes and Genomes (KEGG) pathway analysis was performed based on the SEA with significant altered phosphorylation sites and gene expression with p<0.05 to establish the main affected pathway.

### Data Availability

Microarray data are publicly available from the gene expression omnibus database (https://www.ncbi.nim.nih.gov/geo/).

It will be understood that, while various aspects of the present disclosure have been illustrated and described by way of example, the invention claimed herein is not limited thereto, but may be otherwise variously embodied according to the scope of the claims presented in this and/or any derivative patent application.

### LIST OF ABBREVIATIONS

1,25(OH)2VD3 1,25-dihydroxyvitamin D3 (1,25(OH)2D3)
3D 3-Dimensional
μm Micro Molar
Akt Protein kinase B
CADR Cutaneous Adverse Drug Reactions
Cmax Concentration maximum
DMSO Dimethylsulfoxyde
EGFRi Epidermal Growth Factor Receptor inhibitors
FDR False Discovery Rate
FLG Filaggrin
HER2 Human EGF Receptor 2
IVL Involucrin
KO KEGG orthologs
MDS Multidimensional scaling
nM Nano Molar
PBS Phosphate-Buffered Saline
PDGF Platelet-Derived Growth Factor
PI3K Phosphoinositide 3-kinase
PTEN Phosphatase and tensin homolog
QN Quantile Normalization
RHE Reconstructed Human Epidermis
SD Standard deviation
SEA Set Enrichment Analysis
SIS Stevens-Johnson syndrome
TEN Toxic Epidermal Necrolysis
TKi Tyrosine kinase inhibitors
VD3 vitamin D3 cholecalciferol

### REFERENCES

Ali R, Brown W, Purdy S C, Davisson V J, Wendt M K. Biased signaling downstream of epidermal growth factor receptor regulates proliferative versus apoptotic response to ligand. Cell Death Dis. Springer US; 2018; 9(10) Available from: http://dx.doi.org/10.1038/s41419-018-1034-7
Annunziata M C, Ferrillo M, Cinelli E, Panariello L, Rocco D, Fabbrocini G. Retrospective analysis of skin toxicity in patients under anti-EGFR tyrosine kinase inhibitors: Our experience in lung cancer. Open Access Maced. J. Med. Sci. 2019; 7(6):973-7
Arrieta O, Vega-González M T, López-Macias D, Martinez-Hernández J N, Bacon-Fonseca L, Macedo-Pérez E O, et al. Randomized, open-label trial evaluating the preventive effect of tetracycline on afatinib induced-skin toxicities in non-small cell lung cancer patients. Lung Cancer. Elsevier Ireland Ltd; 2015; 88(3):282-8
Axanova L S, Chen Y Q, McCoy T, Sui G, Cramer S D. 1,25-dihydroxyvitamin D3 and PI3K/AKT inhibitors synergistically inhibit growth and induce senescence in prostate cancer cells. Prostate. NIH Public Access; 2010; 70(15):1658-71
Bakondi E, Gönczi M, Szabó É, Bai P, Pacher P, Gergely P, et al. Role of intracellular calcium mobilization and cell-density-dependent signaling in oxidative-stress-induced cytotoxicity in HaCaT keratinocytes. J. Invest. Dermatol. Blackwell Publishing Inc.; 2003; 121(1):88-95
Bieber T. Atopic dermatitis. N. Engl. J. Med. Massachusetts Medical Society; 2008. p. 1483
Calautti E, Li J, Saoncella S, Brissette J L, Goetinck P F. Phosphoinositide 3-kinase signaling to Akt promotes keratinocyte differentiation versus death. J. Biol. Chem. 2005; 280(38):32856-65
Cheng J B, Levine M A, Bell N H, Mangelsdorf D J, Russell D W. Genetic evidence that the human CYP2R1 enzyme is a key vitamin D 25-hydroxylase. Proc. Natl. Acad. Sci. U.S.A. National Academy of Sciences; 2004; 101(20):7711-5
Dai J, Belum V R, Wu S, Sibaud V, Lacouture M E. Pigmentary changes in patients treated with targeted anticancer agents: A systematic review and meta-analysis. J. Am. Acad. Dermatol. Elsevier Inc; 2017; 77(5):902-910.e2 Available from: http://dx.doi.org/10.1016/j.aad.2017.06.044
Demidenko Z N, Korotchkina L G, Gudkov A V., Blagosklonny M V. Paradoxical suppression of cellular senescence by p53. Proc. Natl. Acad. Sci. U.S.A. National Academy of Sciences; 2010; 107(21):9660-4
Dinour D, Beckerman P, Ganon L, Tordjman K, Eisenstein Z, Holtzman E J. Loss-of-function mutations of CYP24A1, the vitamin D 24-hydroxylase gene, cause long-standing hypercalciuric nephrolithiasis and nephrocalcinosis. J. Urol. J Urol; 2013; 190(2):552-7
Eckert R L, Efimova T, Dashti S R, Balasubramanian S, Deucher A, Crish J F, et al. Keratinocyte survival, differentiation, and death: Many roads lead to mitogen-activated protein kinase. J. Investig. Dermatology Symp. Proc. Blackwell Publishing Inc.; 2002. p. 36-40
Elsholz F, Harteneck C, Muller W, Friedland K. Calcium—A central regulator of keratinocyte differentiation in health and disease. Eur. J. Dermatology. 2014. p. 650-61

Herbig U, Wei W, Dutriaux A, Jobling W A, Sedivy J M. Real-time imaging of transcriptional activation in live cells reveals rapid up-regulation of the cyclin-dependent kinase inhibitor gene CDKN1A in replicative cellular senescence. Aging Cell. Wiley-Blackwell; 2008; 2(6):295-304 Available from: http://doi.wiley.com/10.1046/j.1474-9728.2003.00067.x
Howell M D, Fairchild H R, Kim B E, Bin L, Boguniewicz M, Redzic J S, et al. Th2 cytokines act on S100/A11 to downregulate keratinocyte differentiation. J. Invest. Dermatol. Elsevier
Masson S A S; 2008; 128(9):2248-58 Available from: http://dx.doi.org/10.1038/jid.2008.74
Iglesias-Bartolome R, Patel V, Cotrim A, Leelahavanichkul K, Molinolo A A, Mitchell J B, et al. MTOR inhibition prevents epithelial stem cell senescence and protects from radiation induced mucositis. Cell Stem Cell. NIH Public Access; 2012; 11(3):401-14
Janes S M, Ofstad T A, Campbell D H, Eddaoudi A, Warnes G, Davies D, et al. PI3-kinase dependent activation of apoptotic machinery occurs on commitment of epidermal keratinocytes to terminal differentiation. Cell Res. 2009; 19(3):328-39
Jänicke R U, Sprengart M L, Wati M R, Porter A G. Caspase-3 is required for DNA fragmentation and morphological changes associated with apoptosis. J. Biol. Chem. American Society for Biochemistry and Molecular Biology; 1998; 273(16):9357-60
Joly-Tonetti N, Ondet T, Monshouwer M, Stamatas G N. EGFR inhibitors switch keratinocytes from a proliferative to a differentiative phenotype affecting epidermal development and barrier function. (Under review 2020).
Klaeger S, Heinzlmeir S, Wilhelm M, Polzer H, Vick B, Koenig P-A, et al. The target landscape of clinical kinase drugs. Science. 2017; 358(6367) Available from: http://www.ncbi.nlm.nih.gov/pubmed/29191878
Kroschwald L, Suttorp M, Tabeatauer J, Zimmermann N, Günther C, Bauer A. Off-target effect of imatinib and nilotinib on human Vitamin D3 metabolism. Mol. Med. Rep. Spandidos Publications; 2018; 17(1):1382-8
Lacouture M E. Mechanisms of cutaneous toxicities to EGFR inhibitors. Nat. Rev. Cancer. 2006; 6(10):803-12
Lanning B R, Whitby L R, Dix M M, Douhan J, Gilbert A M, Hett EC, et al. A road map to evaluate the proteome-wide selectivity of covalent kinase inhibitors. Nat. Chem. Biol. 2014; 10(9):760-7
Li D, Ambrogio L, Shimamura T, Kubo S, Takahashi M, Chirieac L R, et al. BIBW2992, an irreversible EGFR/HER2 inhibitor highly effective in preclinical lung cancer models. Oncogene. NIH Public Access; 2008; 27(34):4702-11
Lippens S, Denecker G, Ovaer P, Vandenabeele P, Declercq W. Death penalty for keratinocytes: Apoptosis versus cornification. Cell Death Differ. Nature Publishing Group; 2005; 12(2):1497-508
Marinkovic D, Zhang X, Yalcin S, Luciano J P, Brugnara C, Huber T, et al. Foxo3 is required for the regulation of oxidative stress in erythropoiesis. J. Clin. Invest. American Society for Clinical Investigation; 2007; 117(8):2133-44
Mehlig L M, Garve C, Tauer J T, Suttorp M, Bauer A. Inhibitory effects of imatinib on vitamin D3 synthesis in human keratinocytes. Mol. Med. Rep. Spandidos Publications; 2015; 11(4):3143-7
Missero C, Di Cunto F, Kiyokawa H, Koff A, Dotto G P. The absence of p21 (Cip1/WAF1) alters keratinocyte growth and differentiation and promotes ras-tumor progression. Genes Dev. Cold Spring Harbor Laboratory Press; 1996; 10(23):3065-75
Norlin M, Von Bahr S, Björkhem I, Wikvall K. On the substrate specificity of human CYP27A1: Implications for bile acid and cholestanol formation. J. Lipid Res. 2003; 44(8):1515-22 Nzengue Y, Steiman R, Garrel C, Lefèbvre E, Guiraud P. Oxidative stress and DNA damage induced by cadmium in the human keratinocyte HaCaT cell line: Role of glutathione in the resistance to cadmium. Toxicology. Elsevier; 2008; 243(1-2):193-206
Palmer H G, González-Sancho J M, Espada J, Berciano M T, Puig I, Baulida J, et al. Vitamin D3 promotes the differentiation of colon carcinoma cells by the induction of E-cadherin and the inhibition of β-catenin signaling. J. Cell Biol. J Cell Biol; 2001; 154(2):369-87
Rufini A, Tucci P, Celardo I, Melino G. Senescence and aging: The critical roles of p53. Oncogene. Nature Publishing Group; 2013; 32(43):5129-43 Available from: www.nature.com/onc
Shaurova T, Dy G K, Battaglia S, Hutson A, Zhang L, Zhang Y, et al. Vitamin d3 metabolites demonstrate prognostic value in EGFR-mutant lung adenocarcinoma and can be deployed to oppose acquired therapeutic resistance. Cancers (Basel). 2020; 12(3) Available from: www.mdpi.com/journal/cancers
da Silva Teixeira S, Harrison K, Uzodike M, Rajapakshe K, Coarfa C, He Y, et al. Vitamin D actions in neurons require the PI3K pathway for both enhancing insulin signaling and rapid depolarizing effects. J. Steroid Biochem. Mol. Biol. Elsevier Ltd; 2020; 200:105690
Solca F, Dahl G, Zoephel A, Bader G, Sanderson M, Klein C, et al. Target binding properties and cellular activity of afatinib (BIBW 2992), an irreversible ErbB family blocker. J. Pharmacol. Exp. Ther. American Society for Pharmacology and Experimental Therapeutics; 2012; 343(2):342-50
Teichert A, Bikle D D. Regulation of keratinocyte differentiation by vitamin D and its relationship to squamous cell carcinoma. Signal. Pathways Squamous Cancer. Springer New York; 2011. p. 283-303
Tischer B, Huber R, Kraemer M, Lacouture M E. Dermatologic events from EGFR inhibitors: the issue of the missing patient voice. Support. Care Cancer. Supportive Care in Cancer; 2017; 25(2):651-60 Available from: http://dx.doi.org/10.1007/s00520-016-3419-4
Tsuchisaka A, Furumura M, Hashimoto T. Cytokine regulation during epidermal differentiation and barrier formation. J. Invest. Dermatol. Nature Publishing Group; 2014. p. 1194-6
Vessey D A, Lee K H, Boyer T D. Differentiation-induced enhancement of the ability of cultured human keratinocytes to suppress oxidative stress. J. Invest. Dermatol. Nature Publishing Group; 1995; 104(3):355-8
Vivanco I, Sawyers C L. The phosphatidylinositol 3-kinase-akt pathway in human cancer. Nat. Rev. Cancer. 2002; 2(7):489-501
Wee P, Wang Z. Epidermal growth factor receptor cell proliferation signaling pathways. Cancers (Basel). MDPI AG; 2017. p. 52
Whitman M, Downes C P, Keeler M, Keller T, Cantley L. Type I phosphatidylinositol kinase makes a novel inositol phospholipid, phosphatidylinositol-3-phosphate. Nature. Nature; 1988; 332(6165):644-6
Zhang T, Woods T L, Elder J T. Differential responses of S100A2 to oxidative stress and increased intracellular calcium in normal, immortalized, and malignant human keratinocytes. J. Invest. Dermatol. Blackwell Publishing Inc.; 2002; 119(5):1196-201

1. An in vitro method of evaluating the efficacy of a composition in reducing the effects of a cancer therapeutic on skin, comprising:
employing an in vitro or ex vivo epidermal tridimensional model exhibiting differentiating keratinocytes in a reconstituted stratum corneum;
wherein said model comprises a cancer therapeutic at an amount effective to simulate chronic drug exposure;
wherein said model functionally reproduces barrier compromise due to treatment with said cancer therapeutic; and
wherein said barrier compromise is determined by measuring a resulting biological process.

2. The method of claim 1, wherein the biological process is phosphorylation status of one or more proteins.

3. The method of claim 2, wherein said protein phosphorylation is up-regulated.

4. The method of claim 2, wherein said protein phosphorylation is down-regulated.

5. The method of claim 1, wherein the biological process is gene expression.

6. The method of claim 5, wherein said gene expression is up-regulated.

7. The method of claim 5, wherein said gene expression is down-regulated.

8. Use of the model of claim 1 to test a composition to determine if said composition can prevent the effect said cancer therapeutic has on skin barrier structure and function.
